(19) United States
(12) Patent Application Publication
(10) Pub. No.: US 2004/0082927 A1
(43) Pub. Date: Apr. 29, 2004

(54) Powder dispensing pouch for mens and boys underwear

(76) Inventors: Wade Littleton, Morehead, KY (US); Darrell V. Littleton, Morehead, KY (US)

Correspondence Address: Richard C. Litman, LITMAN LAW OFFICES, LTD., P.O. Box 15035, Arlington, VA, 22215, US

(21) Appl. No.: 10/666,831

(22) Filed: Sep. 12, 2003

Related U.S. Application Data
(60) Provisional application No. 60/421,579, filed on Oct. 28, 2002.

(51) Int. Cl. A61F 5/44; A61F 13/15; A61F 13/20

(52) U.S. Cl. 604/346; 604/359

(57) ABSTRACT

A powder-dispensing pouch that is placed in a fly pocket area of a pair of male underwear. The powder dispensing pouch has a plurality of layers of a porous material joined to create a cavity with a powder contained within the cavity and a sealed edge which keeps the powder between the plurality of layers of the porous material, allowing the powder to be dispersed through the plurality of layers of porous material in a male user's groin area.

### CROSS-REFERENCE TO RELATED APPLICATION

This application claims the benefit of U.S. Provisional Patent Application Serial No. 60/421,579, filed Oct. 28, 2002.

### BACKGROUND OF THE INVENTION

1. Field of the Invention

The present invention relates to a powder-dispensing pouch that is placed in the fly pocket of a pair of mens or boys underwear. Specifically, the invention relates to a powder-dispensing pouch that applies powders to the groin area of a male.

2. Description of Related Art

Many of related art devices utilize male underwear to hold devices that will prevent staining and absorb excess body fluids. These devices are inserted into the underwear next to the male's skin. These devices and other similar devices are reflected in the related art.

U.S. Pat. No. 3,030,958 issued to Levin on Apr. 24, 1962, outlines the use of a structural refinement of a male sanitary garment and is directed more particularly to the provision of a sanitary device for enclosing the private parts of a male person possessing general utility in the related art and having in mind the health and comfort and well being of the wearer.

U.S. Pat. No. 4,826,497 issued to Marcus et al. on May 2, 1989, outlines the use of fibrous absorbent articles such as disposable diapers, catamenial devices, wound dressings, bandages, incontinence pads, wipes, underwear, shoe inserts and the like, that contain an effective amount of crystalline and siliceous material having pore diameters of at least about 5.5 Angstroms and a relatively low capacity for absorbed water.

U.S. Pat. No. 5,392,467 issued to Moretz et al. on Feb. 28, 1995, outlines the use of a moisture-management garment and support garment with a moisture-management panel, which extends from an upper crotch area of the garment to a lower crotch area of the garment. The panel includes an inner fabric layer, an intermediate fabric layer and an outer fabric layer. The inner fabric layer is constructed of wicking yarns for residing in skin contact during garment wear.

U.S. Pat. No. 6,289,520 issued to Page on Sep. 18, 2001, outlines the use of a garment assembly for covering the genitalia of a male user having a perineum adjacently beneath the genitalia. The garment assembly has a front panel member positioned adjacently over the genitalia with the front panel having a top edge, a bottom edge, a first side edge and a second side edge. A suspending mechanism suspends the front panel adjacently over the genitalia with the top edge and the bottom edge is secured to the suspension mechanism.

U.S. Pat. No. 6,409,712 issued to Dutari on Jun. 25, 2002, outlines the use of a disposable garment shield device for men for use with an undergarment for the purpose of protecting the undergarment from staining and soiling due to secretions from the crotch region and genitalia of the user. The shield is constructed of a plurality of layered fabrics or materials designed to wick moisture and excretions away from the skin of the user and retain them in an absorbent layer of the shield.

U.S. Pat. No. 6,447,493 issued to Simpson et al. on Sep. 10, 2002, outlines the use of a thin absorbent patch, similar to an incontinence pad or guard to protect underwear from the leakage of bodily discharges of boys and men. The flexible absorbent article will protect boys and mens underwear from stains caused by excess urine, sexually transmitted diseases, semen and other discharges from the male genitalia.

None of the above inventions and patents, taken either singly or in combination, is seen to describe the instant invention as claimed.

### SUMMARY OF THE INVENTION

The present invention is a powder-dispensing pouch that is placed in a fly pocket area of a pair of male underwear. The powder dispensing pouch has a plurality of layers of a porous material joined to create a cavity with a powder contained within the cavity and a sealed edge which keeps the powder between the plurality of layers of the porous material, allowing the powder to be dispersed through the plurality of layers of porous material in a male user's groin area.

Accordingly, it is a principal object of the invention to provide a powder-dispensing pouch that can be inserted into the fly pocket of a pair of a male's underwear.

It is another object of the invention to eliminate the need for using messy powders to apply to a male's groin area.

It is a further object of the invention to provide a powder-dispensing pouch that can be inconspicuously and comfortably placed in the fly pocket of pair of a male's underwear.

Still another object of the invention is to provide a powder-dispensing pouch that provides long lasting dispensing of a provided powder.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental view showing the placement of a powder-dispensing pouch into a fly pocket of a pair of male's underwear.

FIG. 2 is an overhead perspective view of the powder-dispensing pouch.

FIG. 3 is a cross-sectional perspective view along line 3-3 of FIG. 2.

Similar reference characters denote corresponding features consistently throughout the attached drawings.

### DETAILED DESCRIPTION OF THE PREFERRED EMBODIMENTS

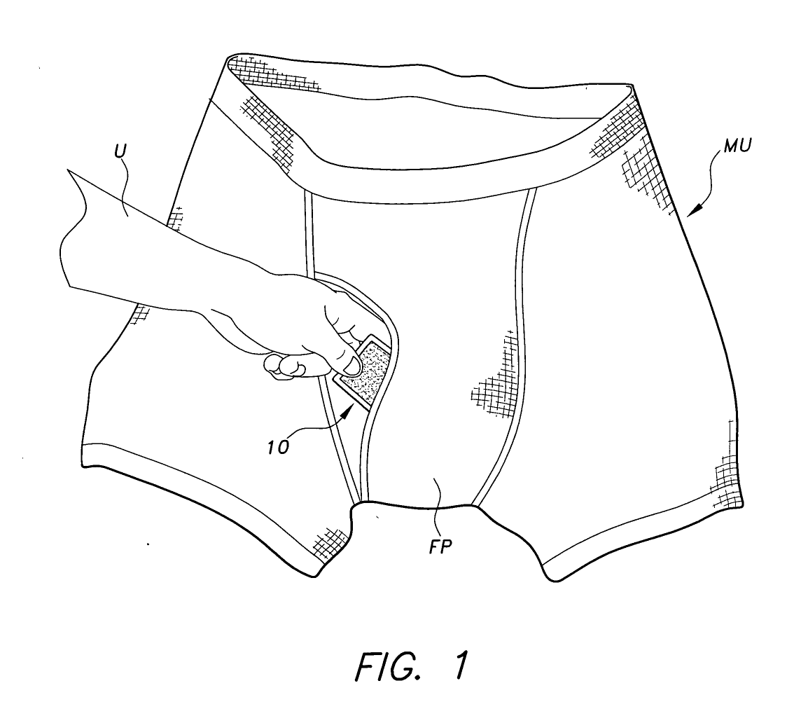

The present invention is a powder-dispensing pouch 10 that is placed in the fly pocket FP area of a male user's U male underwear MU, as is depicted in FIG. 1.

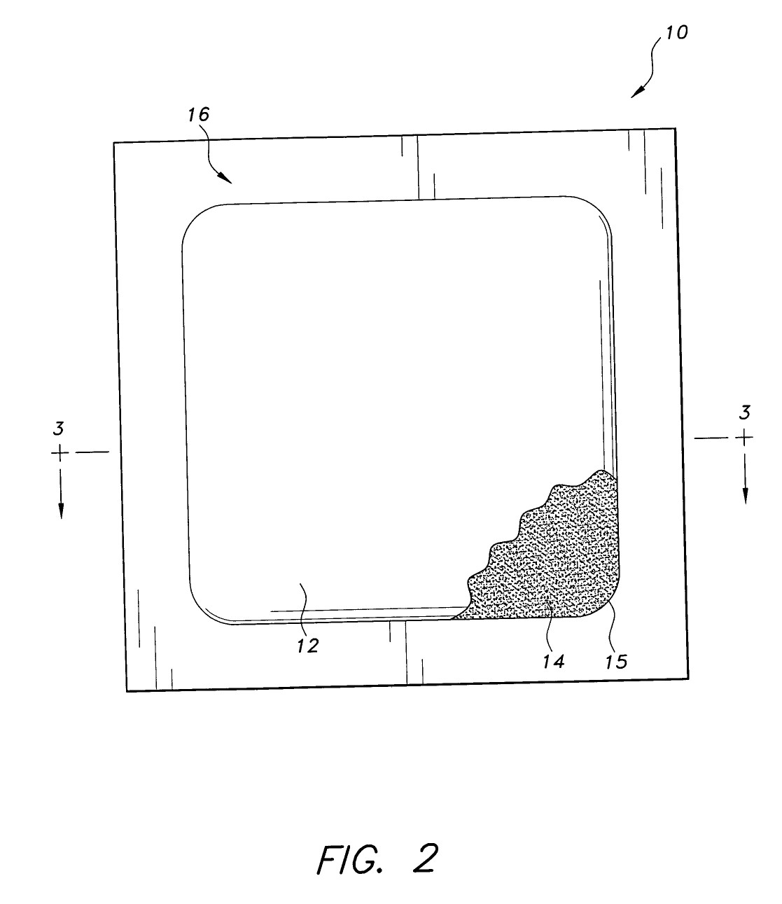

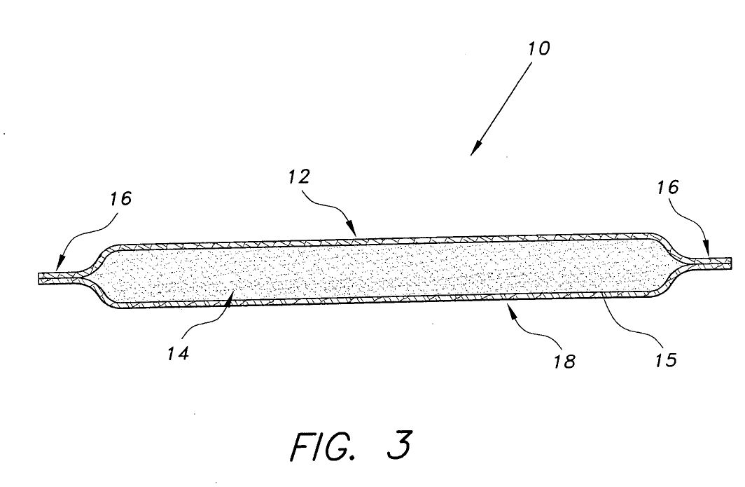

As is shown in FIG. 2 and FIG. 3, the powder-dispensing pouch 10 comprises a plurality of layers of a porous material, which include a top layer of porous material 12 and a bottom layer of porous material 18, which are joined to create a cavity 15. A powder 14 is contained within the cavity 15. The powder-dispensing pouch 10 further comprises a sealed edge 16, which keeps the powder 14 between the top layer of porous material 12 and the bottom layer of porous material 18, allowing the powder 14 to be dispersed through the top layer of porous material 12 and the bottom layer of porous material 18 in a male user's groin area. The top layer of porous material 12 and the bottom layer of porous material 18 can be linen, woven fabric or gauze.

The sealed edge 16 can be created utilizing an adhesive, a heat sensitive adhesive, or the sealed edge can be sewn together with thread. This depends on the type of material used for the two layers 12. This construction creates a cavity into which powder 14 can be held. The type of powder 14 used can be any moisture-absorbing, chafe-reducing, odor-controlling or medicated powder. The powder-dispensing pouch 10 used with male underwear MU must have a fly pocket FP for the powder-dispensing pouch 10 as it is intended.

To use the powder-dispensing pouch 10, a user U obtains a pair of male underwear MU. The powder-dispensing pouch 10 is then inserted into the fly pocket FP of the male underwear MU, where it can settle and stay. The user U then simply wears the male underwear MU as normal. As the user U goes about his daily activities, the powder 14 is slowly dispensed from the powder-dispensing pouch 10. The more the user U moves, the more the powder 14 is released. Any type of male underwear MU that has a fly pocket FP can utilize the powder-dispensing pouch 10.

It is to be understood that the present invention is not limited to the sole embodiments described above, but encompasses any and all embodiments within the scope of the following claims.

We claim:

1. A powder-dispensing pouch that is placed in a fly pocket area of a pair of male underwear, the powder dispensing pouch comprising:
a plurality of layers of a porous material joined to create a cavity;
a powder contained within the cavity;
a sealed edge which keeps the powder between the plurality of layers of a porous material, allowing the powder to be dispersed through the plurality of layers of a porous material in a male user's groin area.

2. The powder-dispensing pouch according to claim 1, wherein the plurality of layers of a porous material include a top layer of porous material and a bottom layer of porous material.

3. The powder-dispensing pouch according to claim 1, wherein the porous material is linen.

4. The powder-dispensing pouch according to claim 1, wherein the porous material is a woven fabric.

5. The powder-dispensing pouch according to claim wherein the porous material is gauze.

6. The powder-dispensing pouch according to claim wherein the powder is moisture-absorbing powder.

7. The powder-dispensing pouch according to claim wherein the powder is chafe-reducing powder.

8. The powder-dispensing pouch according to claim wherein the powder is odor-controlling powder.

9. The powder-dispensing pouch according to claim wherein the powder is medicated powder.
